(19) United States
(12) Patent Application Publication
(10) Pub. No.: US 2011/0089330 A1
(43) Pub. Date: Apr. 21, 2011

(54) Imager

(75) Inventor: Michael Thoms, Bietigheim-Bissingen (DE)

(73) Assignee: DUERR DENTAL AG, Bietigheim-Bissingen (DE)

(21) Appl. No.: 12/936,868

(22) PCT Filed: Mar. 17, 2009
(86) PCT No.: PCT/EP09/01944

§ 371 Date: Dec. 29, 2010

(30) Foreign Application Priority Data
Apr. 11, 2008 (DE) 10 2008 018 598.1

(51) Int. Cl. G01T 1/24

(52) U.S. Cl. 250/370.09

(57) ABSTRACT

The invention relates to an imager, particularly for detecting X-rays, having a sensor sensitive to radiation. The sensor is enclosed in a housing comprising a radiation-permeable inlet window and a cable outlet for a cable arrangement coupled to the sensor. The inlet window comprises a fiber insert embedded in a plastic matrix. The invention is used for imaging methods.

The invention concerns an imager, in particular for detecting X-ray radiation, with a radiation-sensitive sensor, which is surrounded by a housing, which has at least one radiolucent entry window.

An imager which is known from the market, such as is used in particular in the dental field for generating intra-oral X-ray images, includes a CCD sensor, which is held in a liquid-tight housing which is made of a fibre-reinforced synthetic material as an injection-moulded synthetic part. The CCD sensor is connected via a cable arrangement to an analysis device, in which electrical signals of the CCD sensor are analysed. The signals to be analysed are generated by irradiating the CCD sensor with X-ray radiation. Between a source of X-ray radiation and the CCD sensor, an object to be X-rayed, of which the locally different transparency to the provided X-rays results in different exposure of the CCD sensor, can be placed. The signals which the CCD sensor generates can be converted in the analysis device into an image of the object.

For dental use, the housing of the imager must be of stable form, to prevent damage to the CCD sensor when the imager is used, in particular inside the mouth of a patient. Such damage can occur, in particular, if the patient bites on the imager or contact takes place otherwise between the imager and the teeth or jaw of the patient.

In the case of the known imager, the region of the housing called the entry window, through which the X-rays should strike the CCD sensor, like the other regions of the housing, is in a form with thick walls, to ensure the necessary stability.

The object of the invention is to provide an imager which has a more compact form and/or makes improved image quality possible.

This object is achieved by an imager with the features of claim 1.

“Fibre core” should be understood to mean an arrangement of reinforcing fibres, the length of which is calculated so that they do not form an isotropic filler material, but instead are capable of absorbing tensions, like a reinforcement. Such fibres are called “long-fibre” here.

The fibre core is generated in a fibre processing step.

In a processing step downstream from the fibre processing step, the fibre core is embedded in a shapeless mass of thermoplastic or thermosetting synthetic material. The combination of fibre core and mass of synthetic material is then cured, to produce an entry window of stable shape. The curing process can take place by the effect of activation energy and/or by the user of hardener and/or by cooling a heated mass of synthetic material, in particular in a pressing mould or synthetic material injection mould.

What is decisive is that the arrangement of the fibres in the fibre core is at least almost completely fixed before it is embedded in the mass of synthetic material, and is not the result of chance during the flow into the injection mould, as in the case of a conventional injection-moulded synthetic part with filler material with short fibres.

For the fibre core, either an alignment of the fibres in at least one preferred direction or a statistical alignment of the fibres can be provided. Through the predetermined arrangement of the fibres in the fibre core, an alignment of the fibres which ensures the desired high stability of the entry window whereas its thickness is low can be achieved.

In the version of the invention according to claim 2, it is advantageous that the fibres extend over a considerable length of the entry window. Thus, in particular in the case of pointwise loading of the entry window, good load distribution in the fibre core is ensured. The effect of the length of the fibres is that forces acting on the surface of the entry window are distributed over a great fibre length, so that the entry window according to the invention has higher loading capacity. In the case of a rectangular entry window, the mean length of the fibres in the fibre core is at least 20%, preferably at least 40% to 60% of the edge length of the shorter edge of the rectangle. In the case of a curved entry window, the mean length of the fibres in the fibre core is at least 50% of the length of a line laid though the centre of the surface of the entry window to opposite points of the outer edges of the entry window.

In the case of the further development of the invention according to claim 3, it is advantageous that in this way a favourable distribution of forces acting on the entry window can be achieved on a maximum fibre length. In particular if pointwise loading occurs, the tensile forces which occur at the force introduction point onto the fibres are distributed over at least nearly their entire length.

With increasing length of the fibres, the danger of the fibres being torn out of the matrix of synthetic material is reduced.

In the case of the further development of the invention according to claim 4, it is advantageous that the fibre core can be aligned in a separate production step with specifiable properties for the arrangement of the fibres, in particular with respect to preferred directions for the fibres and/or the density of the fibres. Woven fabrics consist of an arrangement of warp threads and woof threads, which are usually aligned perpendicularly to each other. Fleeces consist of undirected fibres, which partly intersect and thus form a flexible compound structure. Fibre non-crimp fabrics consist of fibres lying adjacently and densely parallel to each other.

Preferably, the fibre core according to claim 5 is constructed of multiple individual layers, possibly also of different fibre arrangements. Thus high strength in different directions is obtained.

In a specially preferred way, according to claim 6 multiple layers of fibre arrangements of the fibre core are arranged at an angle to each other with respect to their fibre alignment, so that high strength of the entry window is achieved.

To generate the entry window, after the fibre core is produced, a processing step in which the desired embedding of the fibre core takes place either by impregnating the fibre core with a curable shapeless mass of synthetic material, by laminating the fibre core between synthetic films or by spray-coating the fibre core with a mass of synthetic material in an injection mould, is provided.

In the case of the further development of the invention according to claim 7, it is advantageous that in this way a favourable compromise between the strength of the entry window, the transparency of the entry window to the radiation which is intended to strike the sensor, the material thickness of the entry window and the weight of the entry window is obtained. Carbon fibres have high tensile strength, and reduce the radiation which is intended to strike the sensor only slightly. Thus the entry window according to the invention, compared with traditional entry windows, has increased strength with improved transmission of the radiation which is intended to strike the sensor. Preferably, the dimensioning of the entry window is chosen so that the same strength as in the case of a traditional entry window is present, whereas the transmission for radiation is improved, and a smaller radiation dose, in particular X-ray dose, can be applied to a patient whereas the image quality remains the same. Additionally, because of the significantly increased modulus of elasticity of the entry window according to the invention compared with traditional entry windows, the gap between entry window and sensor can be reduced with no danger of damaging the latter, so that a more compact, in particular thinner, form of the imager can be achieved, since the deflection of the entry window according to the invention under the effect of force is reduced compared with known entry windows.

In the case of the further development of the invention according to claim 8, it is advantageous that by a separating process the entry window can be cut out of a relatively large, in particular commercially available, plate. This can preferably be done by water jet cutting, stamping or laser cutting. Because it is in the form of a flat plate, the entry window has practically identical transmission properties for radiation over its whole extent. It is thus possible to ensure that the rays which enter through the entry window are not subject to inhomogeneous reduction, by which the image quality would be affected.

In the case of the further development of the invention according to claim 9, it is advantageous that in this way a more compact form of the imager can be ensured. It is advantageous if the surfaces of the sensor and entry window are aligned essentially parallel to each other, since then the obtained image is perpendicular to the radiation direction.

In the case of the further development of the invention according to claim 10, it is advantageous that the entry window, being produced by a different method from the rest of the housing, can be generated by a production process which is optimised for its materials and properties.

Preferably, the housing according to claim 11, for attaching the entry window, has a surrounding shoulder, to which the entry window is tightly attached, e.g. using adhesive.

It is specially preferred that according to claim 12, the surrounding shoulder is arranged on the housing in such a way that a surface of the entry window finishes flush with the face of the housing.

Welding the entry window to the housing, in particular using ultrasound welding, is also conceivable.

In another embodiment of the invention, the entry window is received tightly and with positive locking between two frame parts, of which preferably at least one is provided with a surrounding seal, so that the liquid-tightness of the imager in the region of the entry window is ensured.

In the case of the further development of the invention according to claim 13, it is advantageous that between the housing and the entry window, no actions need to be taken for sealing.

Preferably, according to claim 14, the entry window and the housing section are produced from a common fibre core which is impregnated with a mass of synthetic material.

It is specially preferred that according to claim 15, the density of the fibres in the region of the entry window should be chosen to be less than for the rest of the housing, to ensure, in the region of the entry window, advantageous transmission properties for the radiation which is intended to reach the sensor.

According to claim 16, the fibre core for the entry window can be inserted as an insert into an injection mould for synthetic material and spray-coated with synthetic material, by which means a one-piece housing which is reinforced in the region of the entry window can easily be created.

In the case of the further development of the invention according to claim 17, it is advantageous that the thickness of the imager (measured in a cross-section plane perpendicular to the surface of the sensor) can be kept small. A more compact form of the imager results in better handling of the imager, in particular when used in the mouth of a patient.

The further development of the invention according to claim 18 makes it possible to use fibre cores which do not themselves form a light barrier.

Below, an embodiment of the invention is explained in more detail with reference to the drawings, of which:

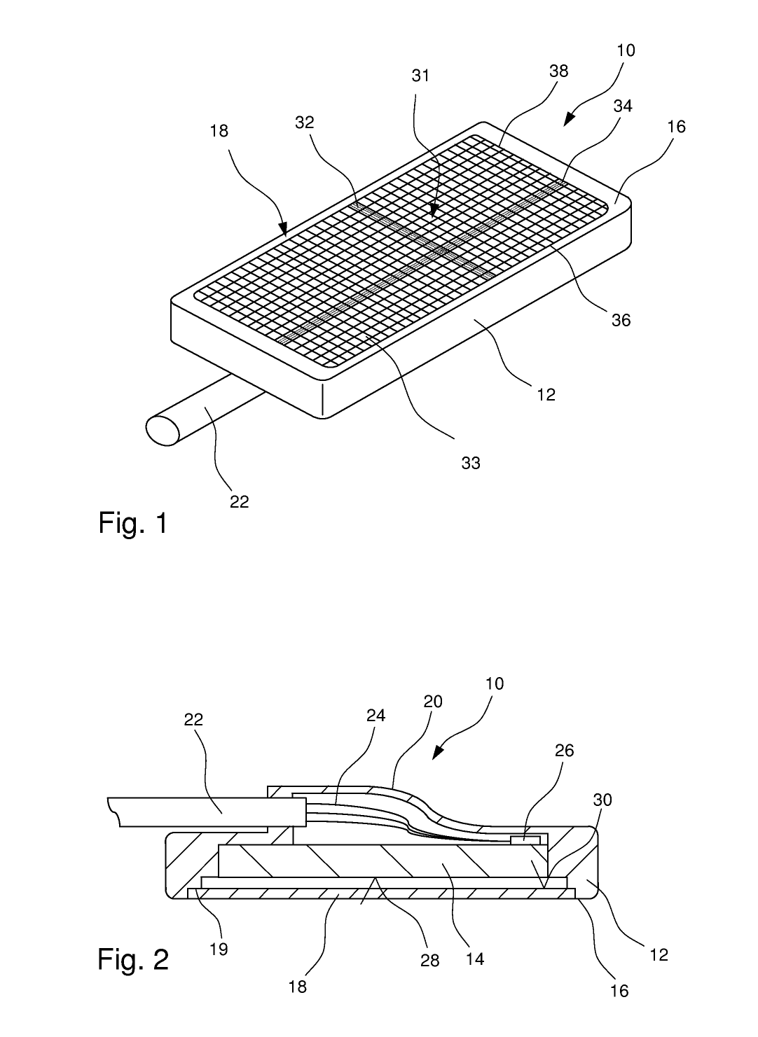

FIG. 1 shows a perspective representation of an imager with an entry window made of carbon fibre composite material, and

FIG. 2 shows a longitudinal section through the imager according to FIG. 1.

An X-ray sensor 10, shown in FIG. 1, includes a housing 12 which is executed as a fibre-reinforced injection-moulded synthetic part, in which housing a CCD sensor 14, which is shown in more detail in FIG. 2, and which is set up to detect X-rays, is arranged.

An object side 16 of the X-ray sensor 10 is provided with an entry window 18 of a carbon fibre composite material. The entry window 18 is on a surrounding shoulder 19 of the housing 12, and is tightly adhesive-bonded to it.

On a surface (the back of the housing 12) facing away from the object side 16, a curved cable output 20, which runs parallel to the longitudinal edge of the housing, is provided centrally, and a connecting cable 22, the cores 24 of which are connected to the CCD sensor 14 in an electrically conducting manner at a connecting area 26, is guided through it. An outlet which is provided on the housing rear wall 40 for the connecting cable 22 is arranged in such a way that the connecting cable leaves the housing 12 essentially parallel to the object side 16.

A front light-sensitive surface 28 of the CCD sensor 14 facing away from the connecting area 26 runs parallel behind the inside 30 of the entry window 18, leaving an air gap, and responds to X-rays.

The carbon fibre composite material of the entry window 18 is, as shown schematically in FIG. 1, executed as a woven fabric 31 of ribbon-like carbon fibre woof threads 32 arranged next to each other and ribbon-like carbon fibre warp threads 34 arranged next to each other, said fabric 31 being embedded in a matrix of synthetic material 33, which is radiolucent to X-ray radiation. The woof threads 32 and warp threads 34 are woven aligned perpendicularly to each other, and run parallel to outer edges 36, 38 of the entry window 18. The length of the woof threads 32 corresponds essentially to the length of the outer edge 38. The length of the warp threads 34 corresponds to the length of the outer edge 36.

To produce the entry window 18, first fabric is cut to the desired size. Multiple pieces of fabric are placed one on top of the other and thus combined into a multilayer fibre core. In a subsequent production step, this is impregnated with epoxy resin, which is cured by the effect of heat in a baking mould. The result is the plate-like and thin-walled shape of the entry window 18.

If carbon fibre woven fabrics are used, the fibre core 31 is usually sufficient to seal off the sensor 14 from external light (daylight). Where less expensive fibre cores, which do not block external light completely, are sufficient to set up the desired mechanical strength, or as additional security, the matrix material of the entry window can also be coloured black, e.g. by an admixture of black pigments.

If a sensor housing including input window is produced from aluminium, there are the following disadvantages:
Al has a higher X-ray absorption than laminated CFC.
The mechanical stability of Al is low compared with laminated CFC (buckling etc. by plastic deformation).
Pointed objects (including teeth in the dental application) in Al easily cause pressure marks and scratches, which can become visible in the image and result in false interpretations (caries).
The sensor becomes heavier if Al is used.
With the same strength, the input window can become thinner, resulting in higher X-ray transmission. In turn a dose saving for the patient follows from this, since the X-ray exposure can be chosen to be lower.
Compared with fibre-reinforced injection-moulded parts or injection-moulded parts of conventional synthetic material, the advantage of the CFC laminate is higher strength and rigidity with the same geometry. In particular, it reliably prevents the possibility of the input window being bent onto the sensitive luminous material layer of the image converter.
Because of the increased flexural strength, the gap between luminous material and input window can be kept smaller, resulting in smaller dimensions.

For illustration, the following mechanical values of carbon components and comparable materials are given:

| | | Tensile | Modulus of | Proportion |
|---|---|---|---|---|
| | Density | strength | elasticity | of fibre |
| Method/material | kg/dm² | N/mm² | N/mm² | % |
| CFC lamination | 1.6 | 500 | 150,000 | 55-65 |
| CFC winding | 1.65 | 1200 | 400,000 | about 70 |
| CFC deep drawing | 1.5 | 500 | 150,000 | 60-65 |
| CFC short fibres | 1.3 | 150 | 12,000 | 20 |
| 1-2 mm | | | | |
| CFC short fibres | 1.4 | 250 | 22,000 | 30 |
| 20-30 mm | | | | |
| Steel | 7.9 | 1000 | 210,000 | |
| Aluminium | 2.7 | 450 | 70,000 | |
| Polyamide | 1.2 | 80 | 1,000 | |
| Epoxy resin | 1.3 | 120 | 3,000 | |

Compared with CFC injection moulding with short fibres, it is seen that the tensile strength in the case of laminated CFC parts is higher by at least the factor 3, and the modulus of elasticity is increased by more than a factor 10, so that significantly higher stability follows. In principle, therefore, the input window can be designed thinner.

Also, because of the much greater rigidity, the risk of damage to the luminous material layer is less. The gap between the input window and the luminous material layer can even be reduced, to reduce the size of the sensor.

Compared with aluminium, a laminated CFC input window has a modulus of elasticity which is approximately twice as high. The above-mentioned advantages are the result.

Another problem in the case of Al is the remaining plastic deformation.

Also, the coefficient of X-ray reduction of Al at 30 keV is about 4 cm⁻¹, whereas that of CFC is about 0.47 cm⁻¹. By choosing laminated CFC, the X-ray transmission of the window material is thus considerably increased compared with aluminium.

Also, by using CFC laminate, in association with the high strength, scratches or craters causing artifacts in the X-ray image because of the stress by teeth, which cannot be avoided in the dental application, is avoided.

Also, the Compton scattering coefficient of aluminium is significantly higher compared with CFC, so that unwanted scattered radiation results the contrast of the X-ray image.

If the input window of CFC laminate is chosen to be of precisely the measurement of the active surface of the sensor behind it, and the adjacent housing is of a different material, the user can easily detect the position of the active surface and place the sensor accordingly. The input window can be mounted by positive locking in a surrounding frame.

According to the invention, CFC laminate can also be used as material for the housing or a housing part of the CCD sensor. Here the region of the input window in front of the active sensor surface can then be delimited optically from the rest of the housing, e.g. by printing. This design has the advantage that the Compton scattering through the housing part also becomes minimal.

If a laminated CFC input window is held in a housing frame of Al, a specially responsive exterior of the sensor is obtained. This construction, which is chosen for optical reasons, cannot then prevent the Compton scattering of the frame; however, it only reduces the contrast at the edge of the input window slightly.

The essential advantages of the invention are thus:
high X-ray transparency of input window,
dose saving,
high mechanical strength,
smaller housing dimensions,
little Compton scattering,
high image contrast,
low weight.

1. An imager for detecting X-ray radiation comprising:
a radiation-sensitive sensor, which is surrounded by a housing, which has at least one radiolucent entry window, wherein the entry window contains a fibre core, which is embedded in a matrix of synthetic material.

2. The imager of claim 1, wherein a mean length of fibres in the fibre core corresponds to at least 20% of an edge length of the entry window.

3. The imager of claim 1, wherein fibres of the fibre core extend essentially over the entry window.

4. The imager of claim 1, wherein the fibre core has at least one fibre arrangement which is formed from fibres, and wherein the fibres are selected from the group consisting of: non-crimp fabric, woven fabric, and fleece.

5. The imager of claim 1, wherein the fibre core is constructed of multiple individual layers.

6. The imager of claim 5, wherein the multiple individual layers are arranged at an angle to each other.

7. The imager of claim 1, wherein the fibre core includes carbon fibres.

8. The imager of claim 1, wherein the entry window is essentially in the form of a flat thin-walled plate.

9. The imager of claim 1, wherein the sensor is arranged in parallel alignment at a short distance of 0.1 to 1 mm, behind the entry window.

10. The imager of claim 1, wherein the entry window is a separate component, and is tightly attached to the housing.

11. The imager of claim 1, wherein the housing has a surrounding shoulder, to which the entry window is tightly attached.

12. The imager of claim 11, wherein the surrounding shoulder is arranged on the housing in such a way that a surface of the entry window is flush with a face of the housing.

13. The imager of claim 1, wherein the entry window is formed in one piece with a housing section which partly surrounds the sensor.

14. The imager of claim 13, wherein the entry window and the housing section which surrounds it are produced from a common fibre core which is impregnated with a mass of synthetic material.

15. The imager of claim 14, wherein a density of the fibres in a region of the entry window is less than a density of fibers for the rest of the housing.

16. The imager of claim 1, wherein the fibre core for the entry window is inserted as an insert into an injection mould for synthetic material and spray-coated with synthetic material therein.

17. The imager of claim 1, wherein a cable outlet is formed on a surface of the housing facing away from the entry window, the cable arrangement leaving the housing essentially parallel to a surface of the entry window.

18. The imager of claim 1, wherein the matrix material of the entry window is in a light-absorbing form.

19. The imager of claim 5, wherein the multiple individual layers are comprised of different fibre arrangements.
